(19) United States
(12) Patent Application Publication
(10) Pub. No.: US 2002/0133127 A1
(43) Pub. Date: Sep. 19, 2002

(54) Self extruding catheter

(76) Inventor: Thomas Collins, Richland, VA (US)

Correspondence Address: Law Office of Frank J. Kowalski, Warrenton Professional Center, Suite 370, 493 Blackwell Road, Warrenton, VA, 20186, US

(21) Appl. No.: 09/805,797

(22) Filed: Mar. 15, 2001

(51) Int. Cl. A61M 5/00

(52) U.S. Cl. 604/264

(57) ABSTRACT

A catheter that is self extruding having its exterior within the interior of the walls as it advances. The catheter includes a tube folded within itself, having an exterior, a first interior, a second interior and a third interior. The catheter advances by having the fold push out through the interior of the catheter to become the exterior of the device. The catheter moves forward by laying down new exterior surface without moving the exterior that is already in place. The interior may be lubricated to facilitate advancement of the catheter while minimizing friction to slow advancement or cause movement of the catheter within the patient. An endoscope may be placed at the distal end of the catheter and move forward as the catheter advances. The catheter may be removed by reversing the process and having the exterior withdraw into the interior, again without any rubbing or other motion of the catheter with respect to the inside of the patient.

### BACKGROUND OF THE INVENTION

1. Field of the Invention

The present invention pertains to catheters and more particularly to non-invasive catheters that enter the patients body and extend into the body without friction causing movement of the catheter itself.

2. Related Prior Art

In modern medical technology, viewing the inside of internal passageways, such as veins or intestines, has become a common procedure. Normally, the tube or catheter is inserted by some means, such as a metal shunt that is inserted into the vein or directly into the intestine. The catheter possibly will have an endoscope or other surgical instrument, such as an everting element or may be used for passage of accessories such as visualization devices, polypectomy snares, cytology brushes, papillotomes, etc. and other devices. The catheter is moved through the internal passage rubbing against the side walls of the vein or intestine. This can cause severe irritation. As a result, many patients can only undergo a one time intestinal exploration because the existing procedures can cause long lasting internal intestine irritation.

Central venous catheters are a special type of intravenous catheter (tube) which are usually inserted in operating room. They are threaded through a vein in the neck (the external or internal jugular vein) or a vein in the upper chest under the collar bone (the subclavian vein) into a large central vein in the chest (the superior vena cava). There are two general types of catheters, those that are permanently placed under the skin with no catheter coming Out through the skin (an internal catheter), or those that come out through the skin (the external catheter).

The major decisions that the individual and health care provider usually face are when and if to place a catheter and which type to use. The placement of a central venous catheter is almost always done as an out-patient procedure with local anesthetic and intravenous sedation. Thus, the minimum amount of discomfort is desired.

There are several different types of catheters. The first type, the midline peripheral catheter type of catheter is inserted into a person's arm near the inside of the elbow and threaded up inside your vein to a length of 6 inches. It is no more painful than having a peripheral IV inserted because one cannot feel the catheter moving up the vein. These catheters typically last about six weeks—a perfect catheter for a short course of antibiotics, but not really practical for long term intravenous therapy. The end of the catheter will reach a much larger vein with more blood flow and will cause less irritation of the vein. Because the catheter is so soft and the end is well inside the vein, the chances of it dislodging are much less than with a peripheral IV.

Another type of catheter is the peripherally inserted central catheter. These catheters are centrally placed, meaning the tip ends up in the Superior Vena Cava, which is the largest vein leading directly to the heart after collecting blood from the rest of the body. “Peripherally inserted” means it goes into your body at your elbow and the tip is threaded up into your vein. Some catheters have a valve at the tip, preventing blood from backing up into the catheter, so heparin is not necessary. Some catheters are usually thinner and more flexible than other types of catheters and do not require a clamp, which adds bulk to the others. These types of catheters are inserted by a nurse, and are usually no more painful than a peripheral IV insertion. After the catheter is inserted, a chest X-ray is required to make sure the tip is in the right location above the heart.

Another type of catheter is the tunnelled central catheter. These catheter tips also end up in the Superior Vena Cava, but the other end is tunnelled about six inches away under the skin on the chest. On the catheter, inside this skin tunnel, is a Dacron cuff which your skin seals around, preventing bacteria from crawling along the outside of the catheter into the blood stream. One type of catheter has valve at the tip, tends to be lighter and more flexible and does not require a bulky clamp. These catheters are surgically placed as an outpatient procedure under local anesthesia. Afterwards, the shoulder area may be somewhat sore for a few days because of the trauma involved. The catheter will usually exit the skin near the nipple area and since the end dangles out, it's always available for use. This makes it very easy to use for daily medications. Though these catheters are designed to be permanent, they are easily pulled out by a doctor with just a slight stinging sensation.

Another type of central catheter is one with implanted ports. Implanted ports have all of the advantages of a central line except they are not always immediately available for use. A port is a small titanium reservoir with a rubber “stopper” that is attached to the catheter entering your vein below the collarbone. The whole thing is implanted under the skin in an outpatient procedure with local anesthesia and IV sedation. The ports are made to withstand 2000 needle entries, but this does not irritate the skin over the port. Most people develop a callus that quits hurting when the needle is placed through the skin. One type of implanted port is actually placed in the arm near the elbow and the catheter line threatened up the vein to the Superior Vena Cava.

Several catheters that are placed in veins are done under local anesthetic. Although there is no pain involved with insertion, there still is some internal damage done when the catheter is inserted and pushed through the vein to its ultimate location. The same type of damage is done when catheters are inserted into the intestines.

Examples of current technology in the area of catheters is demonstrated by the following patents.

U.S. Pat. No. 4,782,819 titled “Optical Catheter”, issued to Edwin L. Adair, relates to a sterilizable catheter of small diameter that has a central coherent fiber bundle for carrying an image to a viewing means and the fiber bundle is surrounded by light fibers. The proximate end of the catheter is provided with a coupling means for aligning the coherent optical bundle with the optical system of the viewing means and for is providing an interface with light transmitting means to transmit to the body cavity. The coupling means may have a diameter no bigger than the diameter of the covering of the catheter. When the catheter is used inside a larger endoscope or a trochar, the endoscope or trochar can be removed while the catheter remains in place. This is accomplished by removing the viewing means and sliding the endoscope or trochar over the catheter and then replacing the viewing means for further viewing within the body cavity. This device can be used for detection for cancer cells and treatment thereof and by phototherapy. A fluorescent dye is attached to the cancer cells and subsequently exposed to an exciting laser light frequency. The fluorescent light is transmitted and displayed on the video monitor and the same light frequency is then transmitted through the light fibers to the cell for phototherapy treatment.

U.S. Pat. No. 5,364,345, titled “Method of Tubal Recanalization and Catheter System Therefor” issued to Guy R. Lowery, et al., relates to a method of tubal recanalization that includes advancing an everting catheter which is adapted for dilatation and an endoscope to a position adjacent the site of a stenosis in an internal tubal passage of a patient. The everting catheter includes an endoscope lumen and the and the endoscope is within the endoscope lumen. The everting element is everted to a location within the stenosis and is then expanded to reduce the stenosis. When the tubal passage has been opened sufficiently, the everting element is retracted. The site of the stenosis is viewed using the endoscope and such viewing may be carried out prior to expansion of the everting element to locate the site as well as after the everting element has been retracted.

U.S. Pat. No. 5,788,681, titled “Multi—Lumen Endoscopic Catheter” issued to George W. Weaver, et al., relates to a multi-lumen catheter intended for advancement through the accessory channel on an endoscope into a body passage into the gastrointestinal system. The catheters have two or more independent lumens extending continuously to ports at the distal top for injection of a contrast medium simultaneously with a guide wire for ERCP procedures and for passage of accessories such as visualization devices, polypectomy snares, cytology brushes, papillotomes and stone baskets for catheterization, diagnosis and treatment within the biliary tract. Use of balloons for maintaining a catheter in fixed position in the biliary tract and for dilatation is also disclosed. The catheters employed are extrusions of a resin comprised of nylon and PEBA. The catheters may also be extruded from polyurethane. Multi-lumen catheters having a reduced diameter distal tip portion on which a dilatation balloon is located are also disclosed. The reduced distal tip portion may serve as a platform for support of a stent.

There are many patents in this area covering all aspects of catheter insertion and advancing. For example, U.S. Pat. No. 4,838,859, titled “Steerable Catheter”, issued to Steve Strassmann, uses inflatable members to direct and steer the tip of the catheter. U.S. Pat. No. 5,938,587, titled “Flexible Inner Liner for the Working Channel of an Endoscope” discloses a catheter with a multiplicity of external ridges on the distal portion of the catheter to reinforce the tubing wall to prevent it from collapsing. However, all the methods and apparatus in the known art must push the distal end forward to advance the catheter to its target.

### SUMMARY OF THE INVENTION

The apparatus and method of the present invention provides a catheter that is self extruding having its exterior within the interior of its walls. The catheter is a flexible tube that is folded within itself, having an exterior with a first interior, a second interior and a third interior. As the catheter is moved forward, the third interior becomes the exterior and the second interior wraps around and becomes the first interior. The distal end rolls out, is set place and becomes part of the exterior of the catheter, being continuously replaced by a new distal end. No part of the exterior of the catheter moves with relationship to the portion of the patient with which it is in contact. Thus, there is little or no irritation using the apparatus and method of the present invention. The catheter advances by having the folded portion of the tube push out through the interior of the catheter to become the exterior of the device. The catheter moves forward by laying down new exterior surface without moving the exterior that is already in place. The interior may be lubricated to facilitate advancement of the catheter while minimizing friction which may slow advancement or cause movement of the catheter within the patient. An endoscope may be placed at the distal end of the catheter and move forward as the catheter advances. The catheter may be removed by reversing the process and having the exterior withdraw into the interior, again without any rubbing or other motion of the catheter with respect to the inside of the patient.

### BRIEF DESCRIPTION OF THE DRAWINGS

FIG. 1 is a plan view of a self extruding catheter according to the design of the present invention.

FIG. 2 is a side sectional view of the self extruding catheter of FIG. 1.

FIG. 3 is a side sectional view of the self extruding catheter of FIG. 1 illustrating its insertion into a vein.

FIG. 4 is a side sectional view of the self extruding catheter of FIG. 1 as may be used with a medical device.

FIG. 5 is a sectional view of a self extruding catheter having a flexible metal guide for aiding advancement of the catheter.

FIG. 6 is an expanded view of the catheter and flexible metal casing of FIG. 5.

### DESCRIPTION OF THE PREFERRED EMBODIMENT

As stated previously, there are several different types of catheters and the type relating to the present invention are those that are inserted into a patient and are guided to a location remote (several inches) from the point of insertion. In traveling through a vein or intestine, prior art catheters rub against the vein or intestine and can cause damage or trauma to the inner walls of the vein or intestine. The present invention avoids any damaging sliding along the inner walls by not advancing in the same manner as prior art catheters.

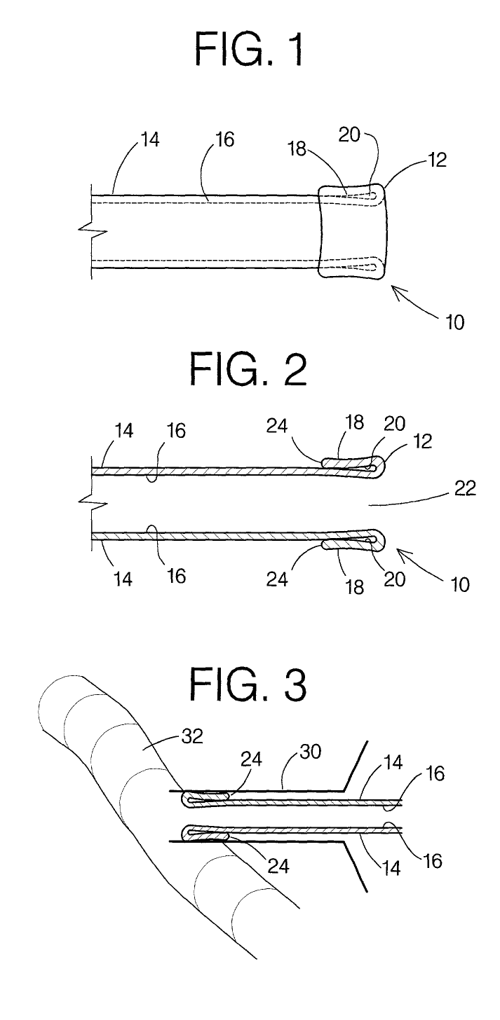

Referring now to FIG. 1, a plan view of a self extruding catheter 10 is illustrated as having a tube 12 with an external outer edge 14 and inner edge 16 (shown in phantom). Once inserted into a patient, inner edge 16 becomes outer edge 18 which contacts the walls of veins or intestines inside the patient and outer edge 14 becomes inner edge 20. As catheter tube 12 advances, it self extrudes by turning itself inside out. As catheter tube 12 advances into the patient, outer edge 18 becomes longer and longer as each portion of edge 18 remains stationary with respect to the wall of the vein or intestine and it lays down additional sections of catheter tubing.

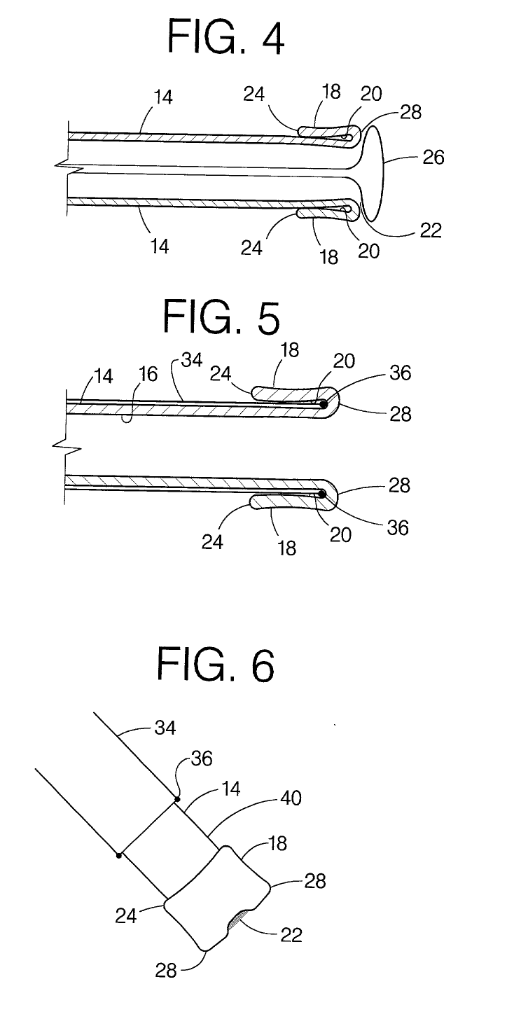

FIG. 2 is a cross sectional view of FIG. 1 illustrating the interior and exterior edges. Interior edge 16 forms an opening 20 through which various medical devices may be inserted into the body of the patient (see FIG. 3). In operation, point 24 of outer edge 18 is fixed (see FIG. 4) so that outer edge 18 remains stationary all along its length. As illustrated, tube 12 is advancing in the patient by being turned inside out. That is, inner edge 16 becomes outer edge 18 as tube 12 moves forward while outer edge 14 becomes inner edge 20 as tube 12 moves forward.

FIG. 3 is a cross sectional view illustrating tube 12 at its initial insertion point into a patient. A metal shunt 26 or needle like device is used to penetrate the skin of the patient and insert into a vein 28. Metal shunt or needle 26 must be securely anchored If to the patient to prevent advancement of needle 26 when catheter 10 advances and to prevent pulling needle 26 out when catheter 10 is withdrawn. With needle 26 pulled out, catheter 10 will not withdraw by reverse extruding but will be puled out possibly causing trauma to the vein or intestine, into whichever it has been inserted.

FIG. 4 is a cross sectional view illustrating self extruding catheter 10 having a medical device 30 within tube 12 and extending slightly from opening 22. Medical device 30 may be of any type known and used in the art, such as an endoscope, a visualization devices, polypectomy snares, a cytology brush, a papillotome, a stone basket for catheterization, etc.. Medical device 30 is arranged to advance simultaneously with tube 12 as it moves forward. In some instances, tube 12 may move medical device 30 forward as tube 12 advances, having a leading edge 32, where the reversal of tube 12 takes place, pushing medical device 30 forward.

FIG. 5 is a cross section view of a self extruding catheter having a flexible stainless steel casing 34 around tube 12 to aid in its advancement. Casing 34 has a smoothed end 36 to glide over outer edge 14 as it reverses direction and becomes inner edge 20. To further aid end 36 in gliding over outer edge 14, a sterile lubricant may be used. A sterile lubricant may be used even when casing 34 is not used. With a lubricant on outer edge 14, then on inner edge 20, the lubricant would not enter the vein or intestine of the patient, but still facilitate advancement of tube 12.

FIG. 6 is an expanded view of FIG. 5 illustrating flexible metal casing surrounding tube 12. In operation, casing 34 is moved up under tube 12 so that it slides under outer edge 18 to point 28 where tube 12 reverses and is laid down inside the vein in which it is located. Casing 34 is used to aid tube 12 in its advancement into the body of the patient. As indicated previously, outer edge 14 may be coated with a sterile lubricant 40 to ease sliding end 36 at the position of reversal of tube 12, point 28. When casing 34 slides over outer edge 14 due to lubricant 40, it will also slide over inner edge 20 since inner edge 20 was outer edge 14 prior to reversal point 28. Thus, an amount of lubricant 40 will be retained by edge 14 as it is turned inside out and becomes inner edge 20.

While there has been illustrated and described a particular embodiment of the present invention, it will be appreciated that numerous changes and modifications will occur to those skilled in the art, and it is intended in the appended claims to cover all those changes and modifications which fall within the true spirit and scope of the present invention.

1. A catheter that is self extruding comprising:
a flexible tube folded within itself having its exterior within the interior of the walls as it advances, said tube having an exterior wall, a first interior wall, a second interior wall and a third interior wall, said second interior wall configured to become said first interior wall as said tube advances and said third interior wall configured to become said exterior wall as said tube advances.

2. The catheter according to claim 1 also including:
a flexible metal casing surrounding said flexible metal tube, said metal casing for advancing said flexible tube.

3. The catheter according to claim 1 also including:
a medical instrument within said flexible tube located at a distal end of said tube.

4. The catheter according to claim 3 wherein said medical instrument is an endoscope.

5. The catheter according to claim 3 wherein said medical instrument is a visualization device.

6. The catheter according to claim 3 wherein said medical device is a polypectomy snares.

7. The catheter according to claim 3 wherein said medical device is a cytology brush.

8. the catheter according to claim 3 wherein said medical device is a papillotome.

9. The catheter according to claim 3 wherein said medical device is a stone basket for catheterization.

10. A method for inserting a catheter into a patient comprising:
providing a tube folded within itself, said tube having an exterior, a first interior, a second interior and a third interior;
advancing said tube into the patient by having the fold push out through the interior of the catheter so that said third interior becomes said exterior of the device and said second interior becomes said first interior; and
moving the catheter forward by laying down a new exterior surface without moving said exterior that is in place.

11. The method according to claim 10 also includes:
lubricating an area between said first interior and said second interior to facilitate advancement of the catheter while minimizing friction to slow advancement or cause movement of the catheter within the patient.

12. The method according to claim 10 also including:
placing an endoscope at a distal end of the catheter; and
moving said endoscope forward as the catheter advances.

13. The method according to claim 10 also including:
removing said catheter by having said exterior withdraw into said interior.
